(19) United States
(12) Patent Application Publication
(10) Pub. No.: US 2015/0239802 A1
(43) Pub. Date: Aug. 27, 2015

(54) HIGH TEMPERATURE REFORMING PROCESS AND CATALYST FOR USE THEREIN

(71) Applicant: UOP LLC, Des Plaines, IL (US)

(72) Inventors: Mark D. Moser, Elk Grove Village, IL (US); Manuela Serban, Glenview, IL (US); Mark P. Lapinski, Aurora, IL (US)

(73) Assignee: UOP LLC, Des Plaines, IL (US)

(21) Appl. No.: 14/187,537

(22) Filed: Feb. 24, 2014

(51) Int. Cl. C07C 2/76

(52) U.S. Cl. CPC C07C 2/76

(57) ABSTRACT

A process for generating aromatics from a hydrocarbon feedstream is disclosed. The process includes the steps of (a) passing the hydrocarbon feedstream to a reformer, wherein the reformer is operated at a temperature greater than 540° C.; and (b) reforming the hydrocarbon feedstream to aromatics in the presence of a catalyst, wherein the catalyst comprises (i) a refractory inorganic oxide support, (ii) a platinum group metal, (iii) a Group IVA metal, (iv) a third metal selected from alkali metals and alkaline earth metals, and (v) a halogen.

### BACKGROUND OF THE INVENTION

The present invention generally relates to combining an alkali/alkaline earth containing reforming catalyst and a high temperature reforming process for the production of aromatic hydrocarbons

### DESCRIPTION OF THE RELATED ART

Catalysts having both a hydrogenation-dehydrogenation function and an isomerization/cracking function (“dual-function” catalysts) are used widely in many applications, particularly in the petroleum and petrochemical industry, to accelerate a wide spectrum of hydrocarbon-conversion reactions. The isomerization/cracking function generally relates to a material of the porous, adsorptive, refractory-oxide type containing an acid function. Typically, this material may be utilized as a support or carrier. The hydrogenation-dehydrogenation function is primarily contributed by a metal component (e.g., Group VIII metals) that is combined with the support.

It is of importance that a dual-function catalyst exhibit the capability both to initially perform its specified functions efficiently and to perform them satisfactorily for prolonged periods of time. The parameters used in the art to measure how well a particular catalyst performs its intended functions in a particular hydrocarbon reaction environment are activity, selectivity and stability. In a reforming environment, these parameters are defined as follows:

Activity is a measure of the ability of the catalyst to convert hydrocarbon reactants to products at a designated severity level representing a combination of reaction conditions: temperature, pressure, contact time, and hydrogen partial pressure. Selectivity refers to the percentage yield of a desired product from a given feedstock at a particular activity level. Stability refers to the rate of change of activity or selectivity per unit of time or of feedstock processed. Activity stability generally is measured as the rate of change of operating temperature per unit of time/feedstock to achieve a given product, with a lower rate of change corresponding to better activity stability.

One process that often employs a dual-function catalyst is catalytic naphtha reforming. Reforming comprises a variety of reaction sequences, including dehydrogenation of cyclohexanes to aromatics, dehydroisomerization of alkylcyclopentanes to aromatics, dehydrocyclization of an acyclic hydrocarbon to aromatics, hydrocracking of paraffins to light products boiling outside the gasoline range, dealkylation of alkylbenzenes and isomerization of paraffins. Some of the reactions occurring during reforming, such as hydrocracking which produces light paraffin gases, are undesirable as they can have a deleterious effect on the yield of a desired product. Improvements in catalytic reforming technology thus are targeted toward enhancing those reactions effecting a higher yield of a desired product.

In some refineries configured for petrochemical production, it may be desirable to carry out additional processing to maximize the yield of valuable xylenes from the aromatic gasoline produced in the reforming process. The xylene isomers are produced in large volumes from petroleum as feedstocks for a variety of important industrial chemicals. Orthoxylene is used to produce phthalic anhydride, which has high-volume but mature markets. Metaxylene is used in lesser but growing volumes for such products as plasticizers, azo dyes and wood preservers. However, the most important of the xylene isomers is para-xylene, the principal feedstock for polyester which continues to enjoy a high growth rate from a large base demand. In addition, often present in xylene mixtures is ethylbenzene, which is occasionally recovered for styrene production, but usually is considered a less desirable component of C₈ aromatics.

The xylenes are not directly recovered from petroleum by the fractionation of naphtha in sufficient volume to meet demand nor in a high enough purity; thus conversion of other hydrocarbons is necessary to increase the purity and yield of the xylenes. For straight run naphtha feedstocks, which may be naphtha distilled out of crude oil, it is necessary to utilize high severity reforming with inter-reactor reheat to convert large amounts of paraffins, such as from about 40 to about 70 weight percent, and having about 30 to about 60% total cyclic content, to the desired xylenes and/or benzene. Moreover, the large amount of non-aromatic content remaining in the reformed naphtha requires substantial subsequent processing to remove the non-aromatics and to transalkylate the aromatics to benzene and xylene.

While the aforementioned dual-function catalysts are capable of catalyzing the dehydrocyclization of paraffins to aromatics such as para-xylene, there is always a trade-off where higher acidity catalysts have more activity but also have reduced selectivity due to increased hydrocracked products, particularly propanes and butanes. Therefore what is needed is a way to eliminate this trade-off where higher selectivity does not come at the cost of lower activity.

### SUMMARY OF THE INVENTION

The inventors have made the surprising discovery that significantly more xylene may be produced in a reforming unit by using reforming catalysts including an alkali and/or alkaline earth metal to reduce the acid cracking of the C8 hydrocarbons and to maximize conversion to xylenes. It has been discovered that operation of the reforming unit in a high temperature regime can improve activity of the aforementioned catalyst while still minimizing cracking reactions. Overall, the combination of an alkali/alkaline earth metal-containing reforming catalyst and a high temperature operating regime has resulted in significant improvements in a reforming process for the production of xylenes and other aromatics.

One embodiment involves a process for generating aromatics from a hydrocarbon feedstream comprising: passing the hydrocarbon feedstream to a reformer, wherein the reformer is operated at a temperature greater than 540° C.; and reforming the hydrocarbon feedstream to generate aromatics in the presence of a catalyst, the catalyst comprising: a refractory inorganic oxide support; a platinum group metal; a Group IVA metal; a third metal selected from the group consisting of alkali metals and alkaline earth metals and mixtures thereof, and a halogen. In one embodiment, the generated aromatics comprise C₈ hydrocarbons; which in turn comprise xylene. The reformer may be operated at a temperature greater than 560° C. The reformer may be operated at a liquid hourly space velocity in a range of 0.6 hr⁻¹ to 10 hr⁻¹ or in a range of 0.6 hr⁻¹ to 5 hr⁻¹. The catalyst may comprise spherical particles comprising (i) the refractory inorganic oxide support, (ii) 0.01 to 2 wt % of the platinum group metal, (iii) 0.01 to 5 wt % of the Group IVA metal, (iv) 0.01 to 1 wt % of the third metal, and (v) 0.1 to 2 wt % of the halogen. The platinum group metal may be platinum. The Group IVA metal may be tin, germanium, or a mixture thereof. The third metal may be cesium, rubidium, potassium, sodium, lithium, calcium, strontium, barium, magnesium and mixtures thereof. Preferably, the third metal is potassium. The refractory inorganic oxide may comprise alumina. The halogen may be chlorine. The metallic elements in the spherical particles may consist essentially of aluminum, platinum, tin, and potassium. The particles may have a diameter of between about 0.7 and about 3.5 millimeters.

In another embodiment the process involves increasing the yield of aromatics when reforming a hydrocarbon feedstream, the process comprising: reforming the hydrocarbon feedstream to generate aromatics at a temperature greater than 540° C. in the presence of a catalyst, the catalyst comprising:(i) a refractory inorganic oxide support, (ii) a platinum group metal, (iii) a Group IVA metal, (iv) a third metal selected from the group consisting of alkali metals and alkaline earth metals and mixtures thereof, and (v) a halogen. The generated aromatics may comprise C₈ hydrocarbons. The catalyst may comprise: spherical particles comprising (i) the refractory inorganic oxide support, (ii) 0.01 to 2 wt % of the platinum group metal, (iii) 0.01 to 5 wt % of the Group IVA metal, (iv) 0.01 to 1 wt % of the third metal, and (v) 0.1 to 2 wt % of the halogen. The inorganic oxide may be alumina, the platinum group metal may be platinum, the Group IVA metal may be tin, the third metal may be potassium, and the halogen may be chlorine.

Yet another embodiment involves increasing the yield of xylenes when reforming a naphtha feedstream by a process comprising: reforming the hydrocarbon feedstream to generate aromatics at a temperature greater than 540° C. in the presence of a catalyst, the catalyst comprising:(i) a refractory inorganic oxide support, (ii) a platinum group metal, (iii) a Group IVA metal, (iv) a third metal selected from the group consisting of alkali metals and alkaline earth metals and mixtures thereof, and (v) a halogen. The catalyst may comprise: spherical particles comprising (i) the refractory inorganic oxide support, (ii) 0.01 to 2 wt % of the platinum group metal, (iii) 0.01 to 5 wt % of the Group IVA metal, (iv) 0.01 to 1 wt % of the third metal, and (v) 0.1 to 2 wt % of the halogen.

These and other features, aspects, and advantages of the present invention will become better understood upon consideration of the following detailed description, drawings and claims.

### BRIEF DESCRIPTION OF THE DRAWINGS

FIG. 1 is a plot of C₃ and C₄ hydrocarbon production as a percent of the total effluent stream from a reforming process as a function of time (hours). HD represents a high density reforming catalyst; LD represents a low density reforming catalyst.

FIG. 2 is a plot of C₈ aromatics production as a percent of the total effluent stream from a reforming process as a function of amount of naphtha feed passed over the catalyst (bbls feed per ft³ catalyst). HD represents a high density reforming catalyst; LD represents a low density reforming catalyst.

FIG. 3 is a plot of xylenes production as a percent of the total effluent stream from a reforming process as a function of amount of naphtha feed passed over the catalyst (bbls feed per ft³ catalyst). HD represents a high density reforming catalyst; LD represents a low density reforming catalyst.

FIG. 4 is a plot of C₉ aromatics production as a percent of the total effluent stream from a reforming process as a function of amount of naphtha feed passed over the catalyst (bbls feed per ft³ catalyst). HD represents a high density reforming catalyst; LD represents a low density reforming catalyst.

FIG. 5 is a plot of C₁₀ aromatics production as a percent of the total effluent stream from a reforming process as a function of amount of naphtha feed passed over the catalyst (bbls feed per ft³ catalyst). HD represents a high density reforming catalyst; LD represents a low density reforming catalyst.

FIG. 6 is a delta plot of para-xylene production as a function of temperature for a series catalysts with varying amounts of potassium. All data is plotted relative to reference Catalyst A, which contains 0 wppm K.

FIG. 7 is a delta plot of C₃ and C₄ hydrocarbon production as a function of temperature for a series catalysts with varying amounts of potassium. All data is plotted relative to reference Catalyst A, which contains 0 wppm K.

### DETAILED DESCRIPTION OF THE INVENTION

As used herein, hydrocarbon molecules may be abbreviated C₁, C₂, C₃ . . . Cₙ, where “n” represents the number of carbon atoms in the one or more hydrocarbon molecules. Cₙ+are hydrocarbons with n or more hydrocarbon atoms. Cₙ−are hydrocarbons with n or fewer hydrocarbon atoms.

As used herein, the terms “alkanes” and “paraffins” may be used interchangeably.

As used herein, the terms “alkenes” and “olefins” may be used interchangeably.

As used herein, the term “weight percent” may be abbreviated as “wt %”.

The present invention uses a catalyst comprising (i) a refractory inorganic oxide support, (ii) a platinum group metal, (iii) a Group IVA metal, (iv) a third metal selected from the group consisting of alkali metals and alkaline earth metals, and (v) a halogen.

The refractory inorganic oxide support usually is a porous, adsorptive, high-surface area support having a surface area of about 25 to about 500 m²/g. Non-limiting example refractory inorganic oxides include alumina, magnesia, titania, zirconia, chromia, zinc oxide, thoria, boria, silica-alumina, silica-magnesia, chromia-alumina, alumina-boria, and silica-zirconia. Preferably, the inorganic oxide refractory support comprises alumina. Suitable alumina materials are the crystalline aluminas known as the gamma-alumina, eta-alumina, and theta-alumina, with gamma-alumina being preferred. The preferred refractory inorganic oxide will have an apparent bulk density of about 0.3 to about 1.0 g/cc and surface area characteristics such that the average pore diameter is about 20 to 300 angstroms, the pore volume is about 0.1 to about 1 cc/g, and the surface area is about 100 to about 500 m²/g.

The preferred form of the catalyst support is a spherical particle, with a preferred diameter of between about 0.7 and about 3.5 millimeters. Alumina spheres may be continuously manufactured by the well known oil-drop method which comprises: forming an alumina hydrosol preferably by reacting aluminum metal with hydrochloric acid; combining the resulting hydrosol with a suitable gelling agent; and dropping the resultant mixture into an oil bath maintained at elevated temperatures. The droplets of the mixture remain in the oil bath until they set and form hydrogel spheres. The spheres are then continuously withdrawn from the oil bath and typically subjected to specific aging and drying treatments in oil and an ammoniacal solution to further improve their physical characteristics. The resulting aged and gelled particles are then washed and dried at a relatively low temperature of about 150° C. to about 205° C. and subjected to a calcination procedure at a temperature of about 450° C. to about 700° C. for a period of about 1 to about 20 hours. This treatment effects conversion of the alumina hydrogel to the corresponding crystalline gamma-alumina.

The platinum group metal comprises platinum, palladium, ruthenium, rhodium, iridium, or osmium, with platinum being preferred. The platinum group metal may exist within the final catalyst as a compound such as an oxide, sulfide, halide, oxyhalide, or as an elemental metal. Best results are obtained when substantially all of the platinum group metal is present in the elemental state. The platinum group metal may be present in the catalyst in any amount which is catalytically effective; the platinum group metal generally will comprise about 0.01 to about 2 wt % of the catalyst, preferably about 0.1 to about 0.4 wt % of the catalyst, and more preferably about 0.2 to about 0.3 wt % of the catalyst.

The platinum group metal may be incorporated in the catalyst in any suitable manner, such as coprecipitation or impregnation. The preferred method of preparing the catalyst involves the utilization of a soluble compound of platinum group metal to impregnate the inorganic oxide support particles in a relatively uniform manner. For example, the platinum group metal may be added to the support by commingling the support with an aqueous solution of chloroplatinic or chloroiridic or chloropalladic acid. Other water-soluble compounds or complexes of platinum-group metals may be employed in impregnating solutions and include ammonium chloroplatinate, bromoplatinic acid, platinum trichloride, platinum tetrachloride hydrate, platinum dichlorocarbonyl dichloride, dinitrodiaminoplatinum, sodium tetranitroplatinate (II), palladium chloride, palladium nitrate, palladium sulfate, diamminepalladium (II) hydroxide, tetramminepalladium (II) chloride, hexamminerhodium chloride, rhodium carbonylchloride, rhodium trichloride hydrate, rhodium nitrate, sodium hexachlororhodate (III), sodium hexanitrorhodate (III), iridium tribromide, iridium dichloride, iridium tetrachloride, sodium hexanitroiridate (III), potassium or sodium chloroiridate, potassium rhodium oxalate, etc. The utilization of a platinum, iridium, rhodium, or palladium chloride compound, such as chloroplatinic, chloroiridic or chloropalladic acid or rhodium trichloride hydrate, is preferred since it facilitates the incorporation of both the platinum group metal component and a quantity of a halogen in a single step. Hydrogen chloride or the like acid is also generally added to the impregnation solution in order to further facilitate the incorporation of the halogen and the metallic components throughout the inorganic oxide support. In addition, it is generally preferred to impregnate the support material after it has been calcined in order to minimize the risk of washing away the platinum group metal.

Of the Group IVA metals in the catalyst, germanium and tin are preferred and tin is most preferred. The Group IVA metal may be present as an elemental metal, as a chemical compound such as the oxide, sulfide, halide, oxychloride, etc., or as a physical or chemical combination with the inorganic oxide support. Preferably, a substantial portion of the Group IVA metal exists in the finished catalyst in an oxidation state above that of the elemental metal. The Group IVA metal optimally is utilized in an amount sufficient to result in a final catalyst including about 0.01 to about 5 wt % of the Group IVA metal, preferably about 0.1 to about 0.5 wt % of the Group IVA metal, and more preferably about 0.2 to about 0.4 wt % of the Group IVA metal.

The Group IVA metal may be incorporated in the catalyst in any suitable manner, such as by coprecipitation with the inorganic oxide support material, ion-exchange with the inorganic oxide support material or impregnation of the inorganic oxide support material at any stage in the preparation. One method of incorporating the Group IVA metal into the catalyst involves the utilization of a soluble compound of a Group IVA metal to impregnate and disperse the metal throughout the inorganic oxide support material. The Group IVA metal can be impregnated either prior to, simultaneously with, or after the other components are added to the inorganic oxide support material. Thus, the Group IVA metal component may be added to the inorganic oxide support material by commingling the inorganic oxide support with an aqueous solution of a suitable metal salt or soluble compound such as stannous bromide, stannous chloride, stannic chloride, stannic chloride pentahydrate. The utilization of Group IVA metal chloride compounds, such as stannic chloride is particularly preferred since it facilitates the incorporation of both the Group IVA metal and an amount of the halogen component in a single step. When combined with hydrogen chloride during the formation of alumina, a homogeneous dispersion of the Group IVA metal component is obtained in accordance with the present invention.

The catalyst includes a third metal selected from the group consisting of alkali metals and alkaline earth metals. The alkali metals are cesium, rubidium, potassium, sodium, and lithium, and the alkaline earth metals are calcium, strontium, barium, and magnesium. Preferably, the third metal is potassium. The third metal optimally is utilized in an amount sufficient to result in a final catalyst including about 0.01 to about 1 wt % of the third metal, preferably about 0.05 to about 0.5 wt % of the third metal, and more preferably about 0.05 to about 0.2 wt % of the third metal. The alkali metal or alkaline earth metal can be incorporated into the inorganic oxide support in various ways with impregnation with an aqueous solution of a suitable water-soluble compound being preferred.

An oxidation step can be used in the preparation of the catalyst. The conditions employed to effect the oxidation step are selected to convert substantially all of the metallic components within the catalyst to their corresponding oxide form. The oxidation step typically takes place at a temperature of from about 370° C. to about 650° C. An oxygen atmosphere is employed typically comprising air. Generally, the oxidation step will be carried out for a period of from about 0.5 to about 10 hours.

In addition to the oxidation step, a halogen adjustment step may also be employed in preparing the catalyst. The halogen adjustment step can serve as a means of incorporating the desired level of halogen into the final catalyst. The halogen adjustment step employs a halogen or halogen-containing compound in air or an oxygen atmosphere. Since the preferred halogen for incorporation into the catalyst comprises chlorine, the preferred halogen or halogen-containing compound utilized during the halogen adjustment step is chlorine, HCl or precursor of these compounds. In carrying out the halogen adjustment step, the catalyst is contacted with the halogen or halogen-containing compound in air or an oxygen atmosphere at an elevated temperature of from about 370° C. to about 650° C. Irrespective of the exact halogen adjustment step employed, the halogen content of the final catalyst should be such that there is sufficient halogen to comprise, on an elemental basis, from about 0.1 to about 5 wt % of the catalyst, preferably about 0.3 to about 2.0 wt % of the catalyst, and more preferably about 0.5 to about 1.5 wt % of the catalyst.

In preparing the catalyst, one can employ a reduction step. The reduction step is designed to reduce substantially all of the platinum group metal component to the corresponding elemental metallic state. Preferably, the reducing gas is substantially pure, dry hydrogen (i.e., less than 20 volume ppm water). However, other reducing gases may be employed such as CO, nitrogen, etc. Typically, the reducing gas is contacted with the oxidized catalyst at conditions including a reduction temperature of from about 315° C. to about 650° C. for a period of time of from about 0.5 to 10 or more hours effective to reduce substantially all of the platinum group metal to the elemental metallic state.

The aforementioned catalysts are beneficially used for reforming of hydrocarbon feedstocks to yield aromatic hydrocarbons such as para-xylene. Suitable hydrocarbon feedstocks include naphtha hydrocarbons.

In regards to use of the catalysts of the present invention in the reforming process, it is desirable to operate the reforming unit within a high temperature regime. A high temperature regime may include temperatures in the range of about 500° C. to about 600° C., and preferably about 540° C. to about 560° C. In one version of the invention, the reformer is operated at a temperature greater than 540° C. In another version of the invention, the reformer is operated at a temperature greater than 550° C. In another version of the invention, the reformer is operated at a temperature greater than 560° C. The advantage of operating the reforming reactor within a high temperature regime relates to the activity of the alkali and/or alkaline earth containing catalysts of the present invention. The addition of, for example, potassium results in increased selectivity for dehydrocyclization but an overall decrease in activity compared to a catalyst without potassium. As a result, increased activity can be obtained by operating the reforming unit at higher temperatures while still maintaining selectivity for conversion to aromatics.

The present invention provides a process that includes the steps of passing the hydrocarbon feedstream to a reformer, wherein the reformer is operated at a temperature greater than 540° C. to generate a process stream comprising aromatic compounds. The process conditions may include a liquid hourly space velocity (i.e., volume of charge per volume of catalyst per hour) in a range of 0.6 hr⁻¹ to 10 hr⁻¹. Preferably, the space velocity in a range of 0.6 hr⁻¹ to 8 hr⁻¹, and more preferably, the space velocity in a range of 0.6 hr⁻¹ to 5 hr⁻¹.

The reforming process is an endothermic process, and to maintain the reaction, the reformer is a catalytic reactor that can comprise a plurality of reactor beds with interbed heaters. The reactor beds are sized with the interbed heaters to maintain the temperature of the reaction in the reactors. A relatively large reactor bed will experience a significant temperature drop, and can have adverse consequences on the reactions. The catalyst can also pass through inter-reformer heaters to bring the catalyst up to the desired reformer inlet temperatures. The interbed heaters reheat the catalyst and the process stream as the catalyst and process stream flow from one reactor bed to a sequential reactor bed within the reformer. The most common type of interbed heater is a fired heater that heats the fluid and catalyst flowing in tubes. Other heat exchangers can be used.

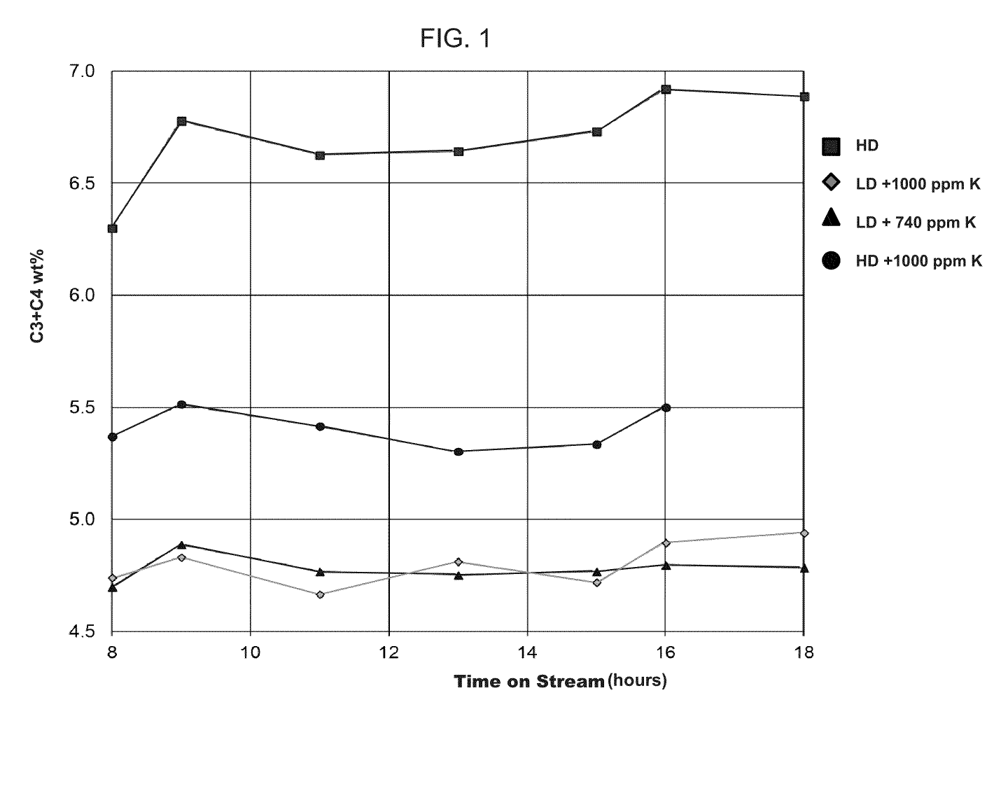

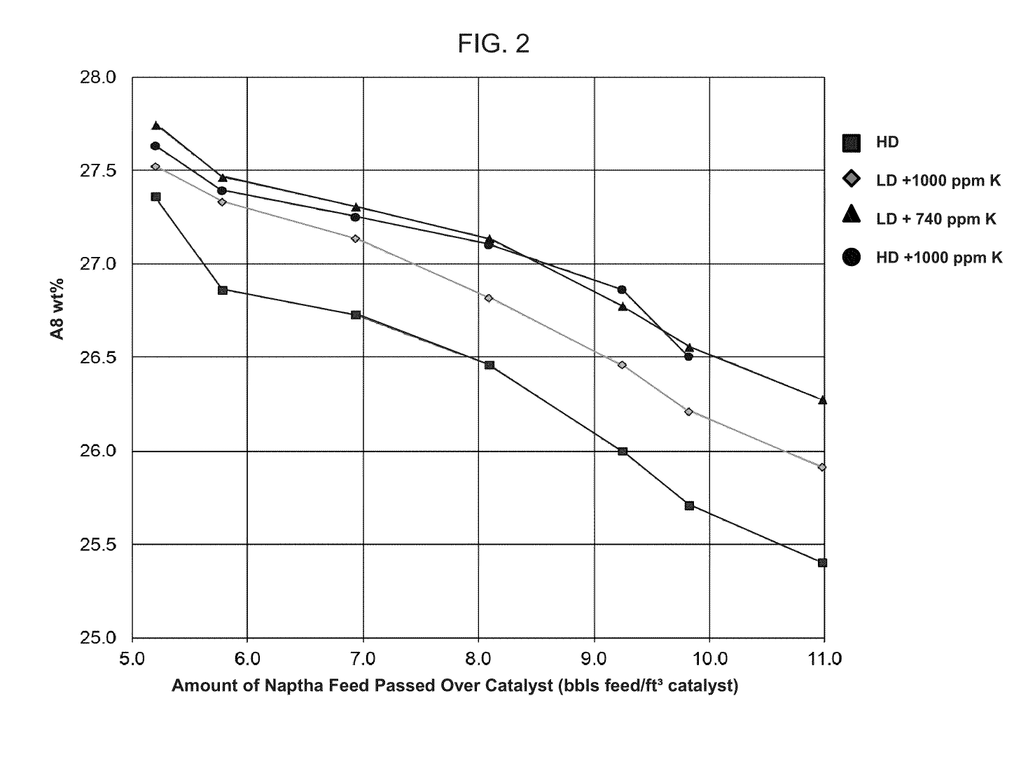

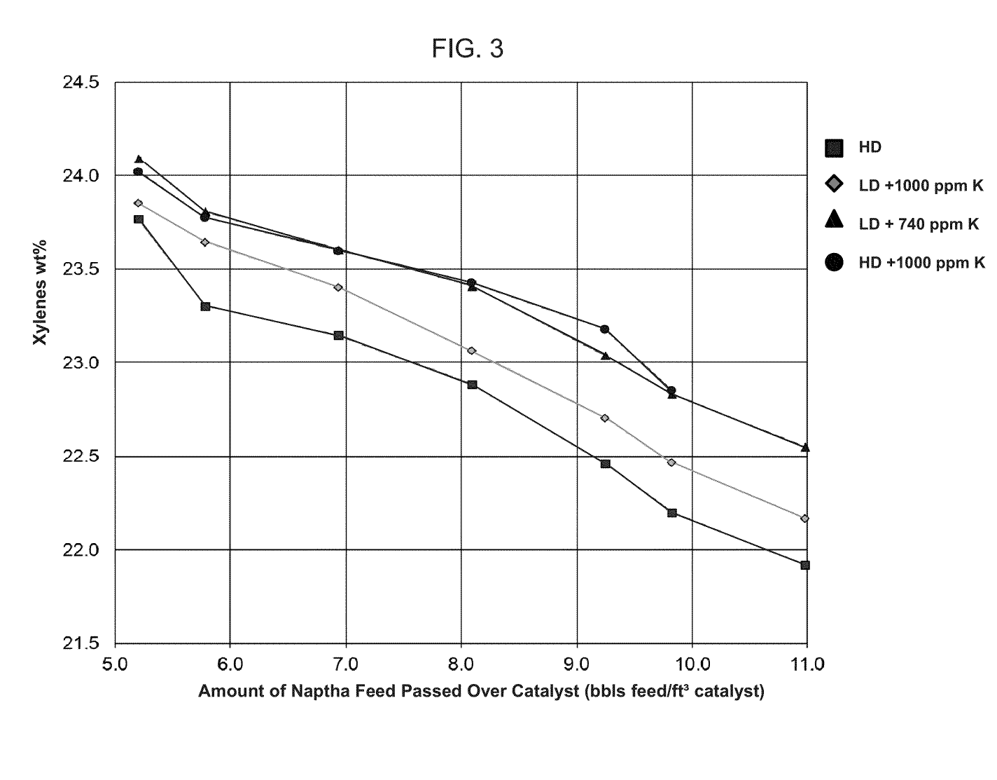

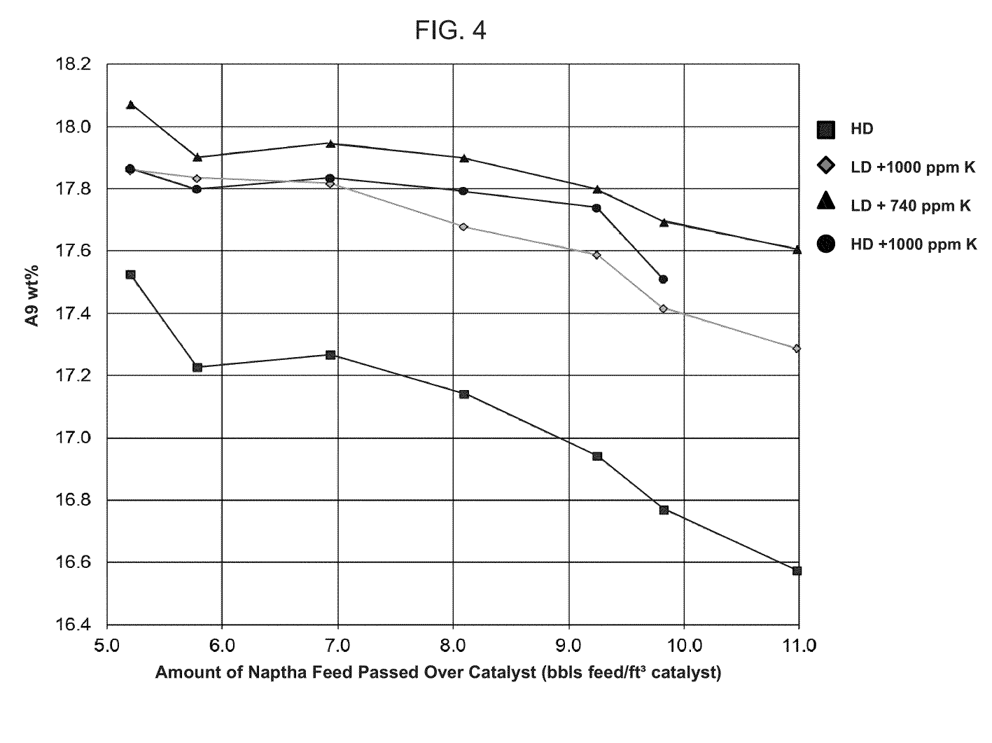

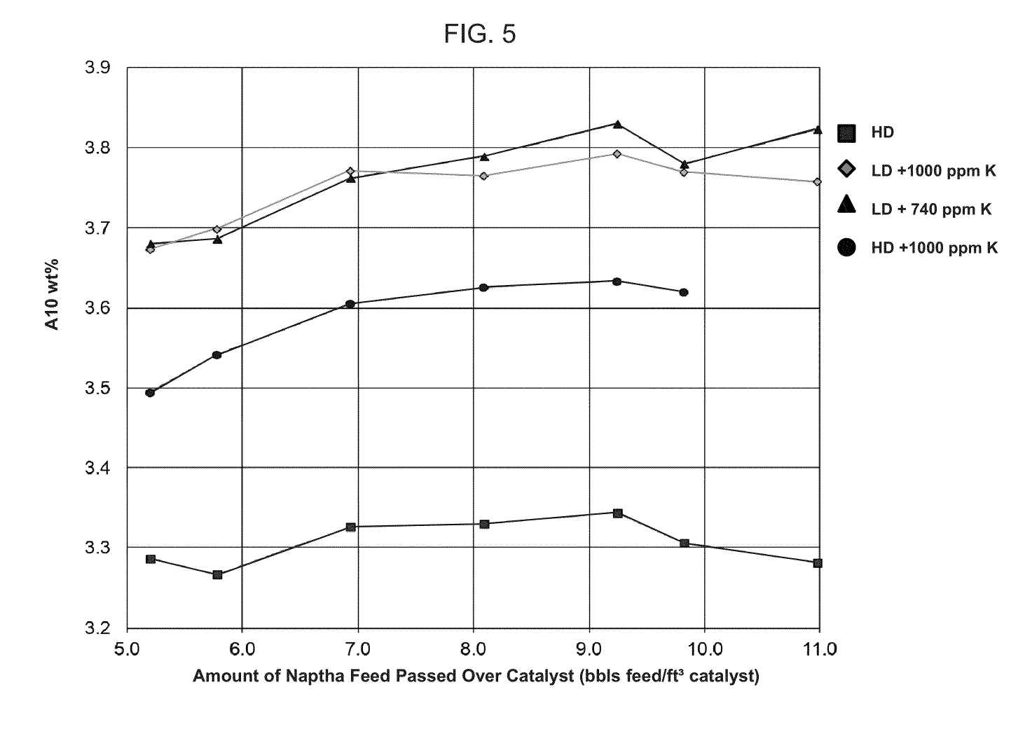

The data, as presented in FIGS. 1-5, shows a significant increase in C₈-C₁₀ aromatics and xylenes and a significant decrease in C₃-C₄ hydrocarbons when the same catalyst includes potassium. The HD catalyst in FIGS. 1-5 included 0.25 wt % Pt, 0.3 wt % Sn, and 1 wt % Cl on a spherical alumina support, and had a density of 0.64 g/cc. The density of the LD catalyst was lower (0.56 g/cc). The ppm of potassium (K) in three of the catalysts are shown in FIGS. 1-6. The catalyst labeled “HD” was tested in the reforming of naphtha at greater than 540° C. and at a liquid hourly space velocity of 3.25. The catalyst labeled “LD+1000 ppm K” was tested in the reforming of naphtha at greater than 540° C. and at a liquid hourly space velocity of 3.25. The catalyst labeled “LD+740 ppm K” was tested in the reforming of naphtha at greater than 540° C. and at a liquid hourly space velocity of 2.5. The catalyst labeled “HD+1000 ppm K” was tested in the reforming of naphtha at greater than 540° C. and at a liquid hourly space velocity of 3.25. The increases in C₈-C₁₀ aromatics and xylenes and the decrease in C₃-C₄ hydrocarbons due to higher temperatures and including potassium in the catalyst allow for increased throughputs, and produce more aromatic products at a lower cost.

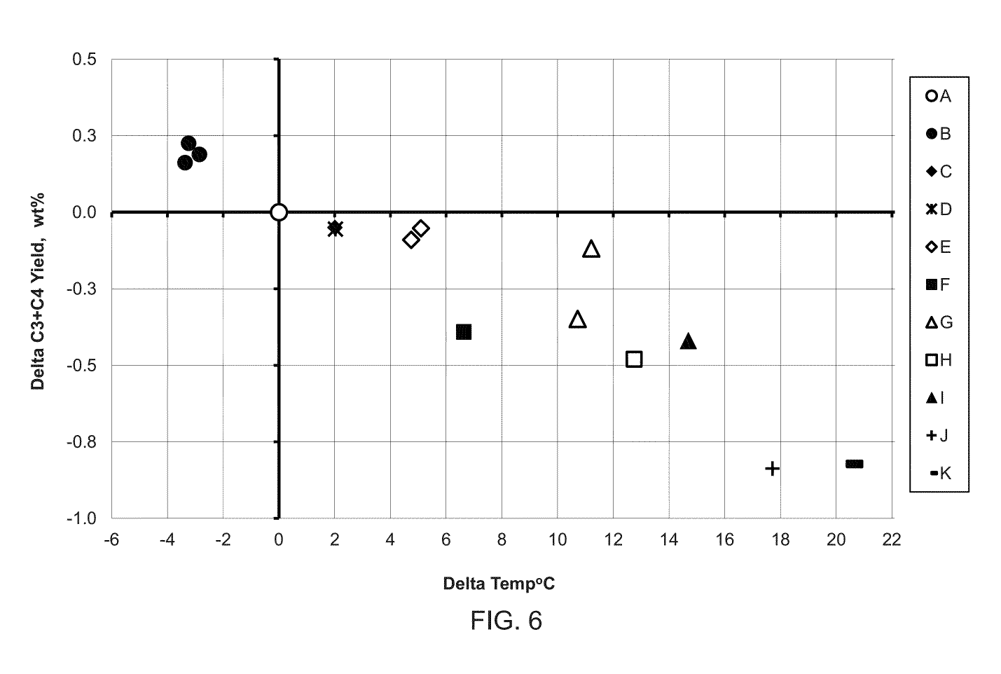

FIG. 6 demonstrates that K-containing samples produced more xylenes than Catalysts A and B that do not contain K. Catalyst A was a high-yield reference catalyst, which is a lanthanide containing spherical alumina catalyst with 0.30 wt % Sn, 0.29 wt % Pt, 1 wt % Cl and 0 wppm K. Catalyst B was a lanthanide-free, spherical alumina catalyst with 0.30 wt % Sn, 0.29 wt % Pt, 1 wt % Cl and 0 wppm K. All the data was plotted relative to

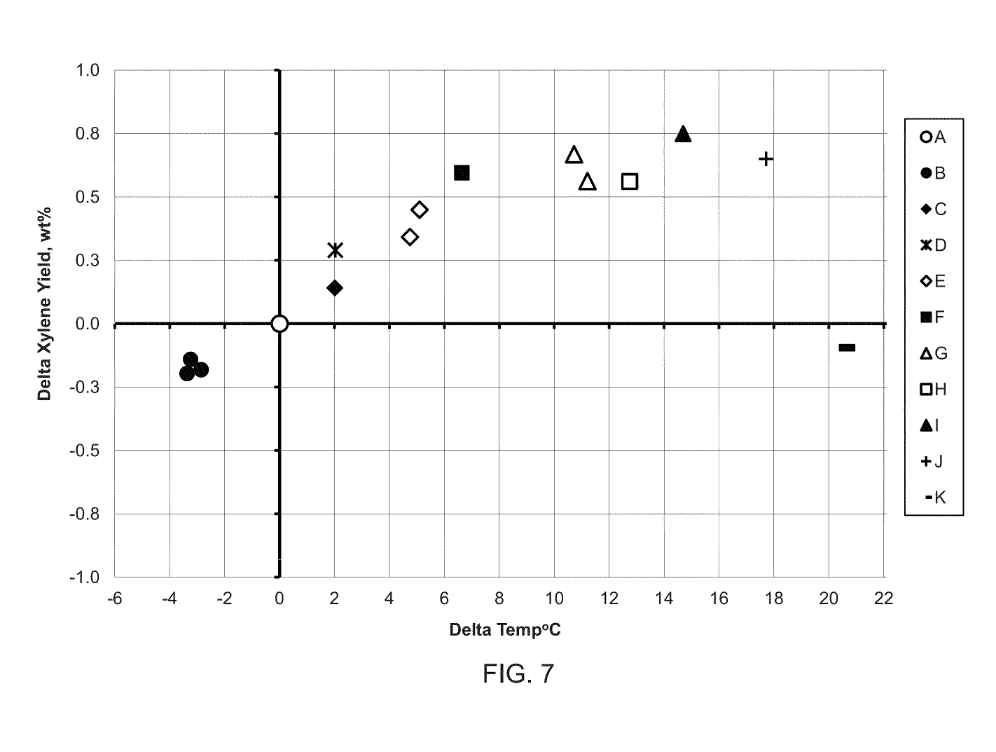

Catalyst A, which corresponds to an absolute yield of 23.5 wt % xylenes from testing a naphtha feed at 513° C. (955° F.) at 1.4 liquid hourly space velocity. The highest xylene yields observed were for Catalyst I with 1100 wppm K operating at a temperature of about 15° C. higher than Catalyst A. The data indicate that at K concentrations over about 1100 wppm K, the xylene yield is decreased. In addition, FIG. 7 shows that C₃+C₄ yields that are produced from undesired acid cracking, were observed to decrease with increasing K concentration. Together, FIGS. 6 and 7 show that the combination of K addition and higher temperatures leads to reduced paraffin cracking to C₃+C₄ products and increased xylene yields with optimal K concentrations observed for a catalyst containing 1100 wppm K. The catalysts of FIGS. 6 and 7 are described in the Table.

TABLE

| Catalyst | wppm K | wt. % Cl |
|---|---|---|
| A | 0 | 1.00 |
| B | 0 | 1.00 |
| C | 210 | 1.00 |
| D | 360 | 0.99 |
| E | 400 | 1.02 |
| F | 530 | 1.04 |
| G | 740 | 0.99 |
| H | 900 | 1.05 |
| I | 1100 | 1.02 |
| J | 1400 | 1.04 |
| K | 2000 | 1.02 |

Thus, the invention provides a process for the production of aromatic hydrocarbons wherein the process may use an alkali/alkaline earth containing reforming catalyst, and a high temperature regime.

Those having skill in the art, with the knowledge gained from the present disclosure, will recognize that various changes could be made in the above embodiments without departing from the scope of the present disclosure. Therefore, the scope of the appended claims should not be limited to the description of the embodiments contained herein.

What is claimed is:

1. A process for generating aromatics from a hydrocarbon feedstream, the process comprising:
(a) passing the hydrocarbon feedstream to a reformer, wherein the reformer is operated at a temperature greater than 540° C.; and
(b) reforming the hydrocarbon feedstream to generate aromatics in the presence of a catalyst, the catalyst comprising:
(i) a refractory inorganic oxide support,
(ii) a platinum group metal,
(iii) a Group IVA metal,
(iv) a third metal selected from the group consisting of alkali metals and alkaline earth metals and mixtures thereof, and
(v) a halogen.

2. The process of claim 1 wherein the generated aromatics comprise C₈ hydrocarbons.

3. The process of claim 1 wherein the generated aromatics comprise xylene.

4. The process of claim 1 wherein the reformer is operated at a temperature greater than 560° C.

5. The process of claim 1 wherein the reformer is operated at a liquid hourly space velocity in a range of 0.6 hr⁻¹ to 10 hr⁻¹.

6. The process of claim 1 wherein the reformer is operated at a liquid hourly space velocity in a range of 0.6 hr⁻¹ to 5 hr⁻¹.

7. The process of claim 1 wherein the catalyst comprises:
spherical particles comprising (i) the refractory inorganic oxide support, (ii) 0.01 to 2 wt % of the platinum group metal, (iii) 0.01 to 5 wt % of the Group IVA metal, (iv) 0.01 to 1 wt % of the third metal, and (v) 0.1 to 2 wt % of the halogen.

8. The process of claim 7 wherein the platinum group metal is platinum.

9. The process of claim 7 wherein the Group IVA metal is tin or germanium.

10. The process of claim 7 wherein the third metal is selected from the group consisting of cesium, rubidium, potassium, sodium, lithium, calcium, strontium, barium, magnesium and mixtures thereof.

11. The process of claim 7 wherein the refractory inorganic oxide comprises alumina.

12. The process of claim 7 wherein the halogen is chlorine.

13. The process of claim 7 wherein the metallic elements in the spherical particles consist essentially of aluminum, platinum, tin, and potassium.

14. The process of claim 7 wherein the particles have a diameter of between about 0.7 and about 3.5 millimeters.

15. A process for increasing the yield of aromatics when reforming a hydrocarbon feedstream, the process comprising:
reforming the hydrocarbon feedstream to aromatics at a temperature greater than 540° C. in the presence of a catalyst, the catalyst comprising:
(i) a refractory inorganic oxide support,
(ii) a platinum group metal,
(iii) a Group IVA metal,
(iv) a third metal selected from the group consisting of alkali metals and alkaline earth metals and mixtures thereof, and
(v) a halogen.

16. The process of claim 15 wherein the aromatics comprise C₈ hydrocarbons.

17. The process of claim 15 wherein the catalyst comprises:
spherical particles comprising (i) the refractory inorganic oxide support, (ii) 0.01 to 2 wt % of the platinum group metal, (iii) 0.01 to 5 wt % of the Group IVA metal, (iv) 0.01 to 1 wt % of the third metal, and (v) 0.1 to 2 wt % of the halogen.

18. The process of claim 17 wherein:
the inorganic oxide is alumina,
the platinum group metal is platinum,
the Group IVA metal is tin,
the third metal is potassium, and
the halogen is chlorine.

19. A process for increasing the yield of para-xylene when reforming a naphtha feedstream, the process comprising:
reforming the hydrocarbon feedstream to aromatics at a temperature greater than 540° C. in the presence of a catalyst, the catalyst comprising:
(i) a refractory inorganic oxide support,
(ii) a platinum group metal,
(iii) a Group IVA metal,
(iv) a third metal selected from the group consisting of alkali metals and alkaline earth metals and mixtures thereof, and
(v) a halogen.

20. The process of claim 19 wherein the catalyst comprises:
spherical particles comprising (i) the refractory inorganic oxide support, (ii) 0.01 to 2 wt % of the platinum group metal, (iii) 0.01 to 5 wt % of the Group IVA metal, (iv) 0.01 to 1 wt % of the third metal, and (v) 0.1 to 2 wt % of the halogen.
